(19) United States
(12) Patent Application Publication
(10) Pub. No.: US 2025/0325228 A1
(43) Pub. Date: Oct. 23, 2025

(54) ILLUMINANCE MEASURING APPARATUS, BIO-ILLUMINANCE MEASURING SYSTEM, AND BIO-ILLUMINANCE MEASUREMENT METHOD USING THE SAME

(71) Applicant: KOOKMIN UNIVERSITY INDUSTRY ACADEMY COOPERATION FOUNDATION, Seoul (KR)

(72) Inventors: Hyun Sun MO, Seoul (KR); Dae Jeong KIM, Seoul (KR)

(21) Appl. No.: 19/253,807

(22) Filed: Jun. 28, 2025

Related U.S. Application Data
(63) Continuation of application No. PCT/KR2023/020923, filed on Dec. 19, 2023.

(30) Foreign Application Priority Data
Dec. 29, 2022 (KR) 10-2022-0188321

(51) Int. Cl. A61B 5/00

(52) U.S. Cl. CPC A61B 5/4857; A61B 5/0059; A61B 2562/0233

(57) ABSTRACT

Disclosed is a system for measuring bio-illuminance. A system for measuring bio-illuminance according to an embodiment of the present disclosure includes: an analog front-end chip including a first photosensor and a second photosensor; a transceiving device; and at least one processor configured to calculate bio-illuminance, in which the analog front-end chip is configured to sense external light and output a first photovoltage and a second photovoltage as digital data, respectively, using the first photosensor and the second photosensor, the transceiving device is configured to wirelessly or wiredly transmit the digital data of the first photovoltage and the second photovoltage to the processor, and the at least one processor is configured to calculate the bio-illuminance and output the calculated bio-illuminance by invoking a pre-stored function based on the first photovoltage and the second photovoltage.

### TECHNICAL FIELD

The present disclosure relates to an apparatus, a system, and a method for measuring bio-illuminance and color temperature, and particularly, to a bio-illuminance measuring apparatus, a bio-illuminance measuring system, and a bio-illuminance measurement method using the same, which are capable of calculating bio-illuminance and color temperature without a spectrometer.

### BACKGROUND ART

The human circadian rhythm is influenced by light. When the circadian rhythm is disturbed due to improper light exposure, hormones are affected and there is an increasing probability of exposure to various diseases such as seasonal emotional disorders, sleep disorders, depression, jet lag fatigue, and health diseases associated with shift work increases. For this reason, it is necessary to measure bio-luminance, which is a light index that affects the circadian rhythm. By measuring the bio-illuminance, it is possible to diagnose the user's light exposure and light contamination, and it is possible to seek a method capable of enhancing the user's circadian rhythm according to the diagnosis result.

The bio-illuminance is related to the intensity of light, i.e., illuminance (Lux) and circadian action factor (CAF). The circadian action factor (CAF) is one of the light source characteristic indicators according to a spectral composition of a light source, and can be utilized along with another characteristic indicator, such as a correlated color temperature (CCT). While the correlated color temperature (CCT) is widely used as an indicator that affects human sensibility, the circadian action factor (CAF) is a better indicator for an awakening promoting effect of light. That is, the higher the circadian action factor (CAF), the higher the level of awakening of the human body.

However, in order to obtain such information, it is necessary to use an expensive spectrometer, and the spectrophotometer has difficulty in miniaturization due to characteristics of a sensor and a detector, and thus new types of bio-illuminance measuring apparatus, bio-illuminance measuring system, and bio-illuminance measurement method have been required.

### SUMMARY

### Technical Problem

An object to be achieved by the present disclosure is to provide a bio-illuminance measuring apparatus, a bio-illuminance measuring system, and a bio-illuminance measurement method using the same based on a correlated color temperature (CCT) and a circadian action factor (CAF).

The objects to be achieved by the present disclosure are not limited to the aforementioned objects, and other objects, which are not mentioned above, will be apparently appreciated by those skilled in the art from the following description.

### Technical Solution

In order to achieve the above-described object, a system for measuring bio-illuminance according to an embodiment of the present disclosure includes: an analog front-end chip including a first photosensor and a second photosensor; a transceiving device; and at least one processor configured to calculate bio-illuminance, and the analog front-end chip is configured to sense external light, and output a first photovoltage and a second photovoltage as digital data, respectively, using the first photosensor and the second photosensor, the transceiving device is configured to wirelessly or wiredly transmit the digital data of the first photovoltage and the second photovoltage to the processor, and the at least one processor is configured to calculate the bio-illuminance and output the calculated bio-illuminance by invoking a pre-stored function based on the first photovoltage and the second photovoltage.

In order to achieve the above-described object, a method for measuring bio-illuminance according to an embodiment of the present disclosure includes: sensing external light using a first photosensor and a second photosensor; converting a first photovoltage and a second photovoltage respectively generated by the first photosensor and the second photosensor into digital data, respectively; transmitting the digital data for the first photovoltage and the second photovoltage to a pre-stored function; and calculating bio-illuminance by inputting the transmitted first photovoltage and second photovoltage data into the pre-stored function.

In order to achieve the above-described object, an apparatus for measuring bio-illuminance according to an embodiment of the present disclosure includes: an analog front-end chip including a first photosensor and a second photosensor having different light response characteristics, and configured to output digital data for a first photovoltage and a second photovoltage using the first photosensor and the second photosensor; at least one processor configured to calculate bio-illuminance; and a transceiving device configured to wirelessly or wiredly transmit the digital data for the first photovoltage and the second photovoltage to the processor, and the at least one processor is configured to calculate a ratio of the first photovoltage and the second photovoltage, calculate a correlated color temperature of external light and a circadian action factor of the external light based on the ratio, calculate illuminance based on the first photovoltage and the correlated color temperature, and calculate the bio illuminance based on the illuminance and the circadian action factor.

Besides, a computer-readable recording medium having a computer program for executing another method, another apparatus, another system, and another method for implementing the present disclosure recorded therein may be further provided.

### Advantageous Effects

According to various embodiments of the present invention as described above, the present invention can measure illuminance, a color temperature, a circadian action factor, and bio illuminance in a relatively simple scheme by utilizing digital data measured from two photosensors having different light response characteristics.

Since the present disclosure can omit additional components for calculating bio-illuminance, such as a circadian wavelength filter and a visual wavelength filter, by integrating a photosensor in a chip, there are effect in that it is possible to downsize a bio-illuminance measuring apparatus, and the present disclosure can be applied to various products at low cost.

According to the present disclosure, an accurate bio-illuminance value can be calculated by detecting optical data in a single chip in which the photosensor is embedded.

In addition, according to the present disclosure, the present disclosure can be utilized as a method for diagnosing a degree of light exposure and light contamination related to a circadian rhythm by indicating a level of awakening of a human body, as well as expressing a concentration and an emotional level by illumination by sequentially computing and calculating the color temperature, illuminance, circadian action factor, and bio-illuminance.

The effects of the present disclosure are not limited to the aforementioned effect, and other effects, which are not mentioned above, will be apparently appreciated by those skilled in the art from the following disclosure.

### BRIEF DESCRIPTION OF DRAWINGS

FIG. 1 is a diagram illustrating a relative sensitivity curve according to a wavelength according to an embodiment of the present disclosure.

FIG. 2 is a diagram for schematically describing a bio-illuminance measuring system according to an embodiment of the present disclosure.

FIG. 3 is a diagram illustrating light response characteristics of a photosensor according to an embodiment of the present disclosure.

FIG. 4 is a diagram illustrating a sensitivity curve of the photosensor according to an embodiment of the present disclosure.

FIG. 5 is a diagram illustrating an operation of a bio-illuminance calculation server according to an embodiment of the present disclosure.

FIG. 6 is a diagram illustrating a color temperature and a voltage ratio in accordance with a change in illuminance according to an embodiment of the present disclosure.

FIG. 7 is a diagram illustrating a correlation between a photovoltage and illuminance in accordance with the color temperature according to an embodiment of the present disclosure.

FIG. 8 is a diagram illustrating a correlation function group of the photovoltage and the illuminance according to an embodiment of the present disclosure.

FIG. 9 is a diagram illustrating a correlation between a circadian action factor and a voltage ratio according to an embodiment of the present disclosure.

FIG. 10 is a diagram illustrating a correlation between bio-illuminance and illuminance in accordance with the color temperature according to an embodiment of the present disclosure.

FIG. 11 is a diagram for schematically describing a bio-illuminance measuring system according to an embodiment of the present disclosure.

FIG. 12 is a diagram for schematically describing a bio-illuminance measuring system according to an embodiment of the present disclosure.

FIG. 13 is a flowchart illustrating a bio-illuminance measurement method according to an embodiment of the present disclosure.

FIG. 14 is a flowchart illustrating a bio-illuminance measurement method according to an embodiment of the present disclosure.

FIG. 15 is a flowchart illustrating a bio-illuminance measurement method according to an embodiment of the present disclosure.

### DETAILED DESCRIPTIONS

Effects and features of the present disclosure, and methods for accomplishing the same will be apparent with reference to embodiments described in detail below along with the accompanying drawings. However, the present disclosure is not limited to embodiments disclosed below but may be implemented in various different forms.

The terminology used herein is for the purpose of describing embodiments of the present disclosure and the present disclosure is not limited to the terminology used in the specification. Unless otherwise specified herein, the singular forms include plural forms as well. The terms “comprise” and/or “comprising” used in the specification does not exclude the presence or addition of one or more other components other than stated components. Like reference numerals refer to like components throughout the specification and “and/or” includes respective mentioned components and all one or more combinations of the components. The terms ‘first’, ‘second’, etc. are used herein to distinguish one component from another and/or to describe a plurality of components, but the components are not limited by these terms. Therefore, a first component to be mentioned below may be a second component within the technical spirit of the present disclosure.

Unless otherwise defined, all terms including technical and scientific terms used in the present specification may be used as the meaning which may be commonly understood by the person with ordinary skill in the art, to which the present disclosure pertains. Further, terms used herein and defined by commonly used dictionaries are not to be construed ideally or unduly unless expressly specifically defined herein.

Hereinafter, embodiments of the present disclosure will be described in detail with reference to the accompanying drawings.

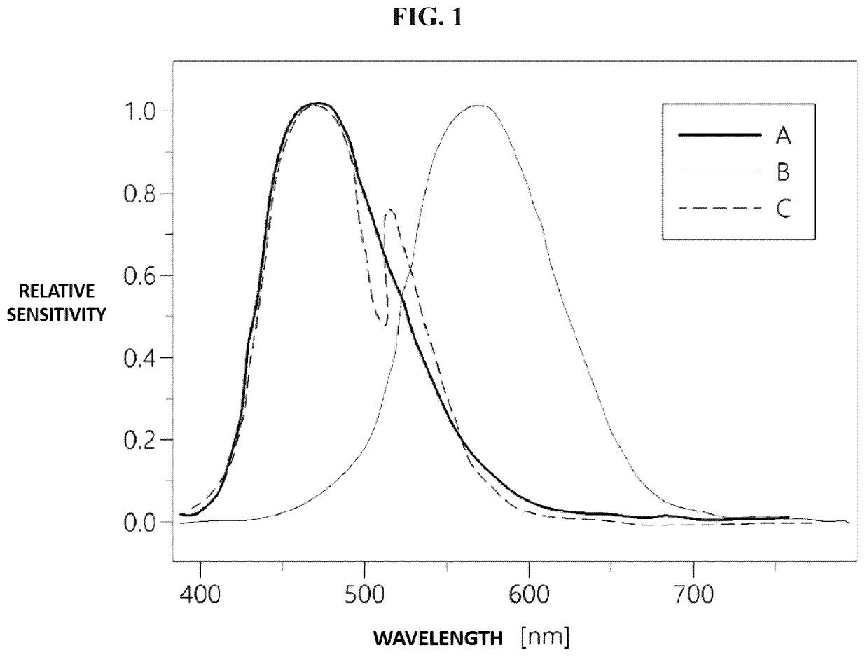

FIG. 1 is a diagram illustrating a relative sensitivity curve according to a wavelength according to an embodiment of the present disclosure.

Referring to FIG. 1, a circadian sensitivity curve A is a light sensitivity characteristic curve for a hormone that governs a human circadian rhythm, and is a curve having maximum sensitivity in a circadian wavelength band. The hormones may include melatonin, cortisol, and the like. The circadian wavelength band may be 400 nm to 600 nm. A circadian wavelength filter following the circadian sensitivity curve may operate as a bandpass filter that passes external light having a wavelength within 400 nm to 600 nm which is the circadian wavelength band and blocks external light having a wavelength band other than the circadian wavelength band.

A visual sensitivity curve B as a light sensitivity characteristic curve for a human eye is a curve that has maximum sensitivity in a visual wavelength band. The visual wavelength band may be 450 nm to 700 nm. A visual wavelength filter following the visual sensitivity curve B may operate as a bandpass filter that passes external light having a wavelength of 450 nm to 700 nm, and blocks external light having a wavelength band other than the wavelength of 400 nm to 700 nm.

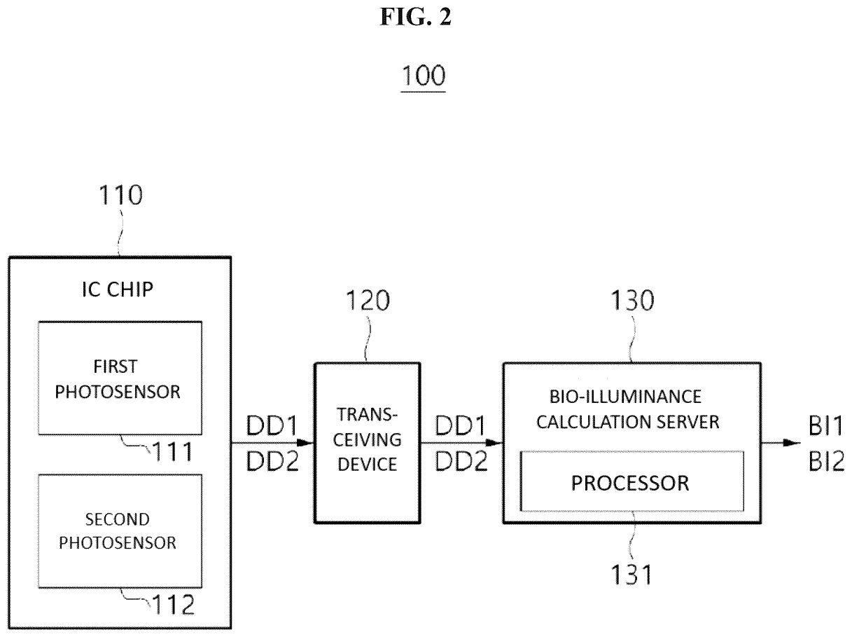

A general illuminance (Lux) measuring apparatus measures illuminance of external light using the visual wavelength filter following the visual sensitivity curve B of FIG. 2. The illuminance may refer to the intensity of light that may be perceived by the human eye, and may have a maximum sensitivity at a wavelength of 380 nm to 780 nm. Further, a general bio-illuminance (Biolux) measuring apparatus measures bio-illuminance using the circadian wavelength filter following the circadian sensitivity curve A of FIG. 2. According to an embodiment, both the circadian wavelength filter and an illuminance wavelength filter may be used.

A curve C extracted according to an embodiment of the present disclosure may have characteristics of both the circadian sensitivity curve A and the visual sensitivity curve B. For example, a wavelength band of the curve C may be 400 nm to 700 nm. The curve C may be a sensitivity curve that is extracted as the present disclosure utilizes two photosensors. According to the present disclosure, as two photosensors are used, the bio-illuminance may be measured without using the visual wavelength filter and the circadian wavelength filter.

As a result, since the present disclosure can omit additional components for calculating bio-illuminance, such as a circadian wavelength filter and a visual wavelength filter, by integrating a photosensor in a chip, it is possible to downsize a bio-illuminance measuring apparatus, and the present disclosure can be applied to various products at low cost. In addition, according to the present disclosure, an accurate bio-illuminance value may be calculated by detecting optical data in a single chip in which the photosensor is embedded, and the present disclosure can be utilized as a method for diagnosing a degree of light exposure and light contamination related to a circadian rhythm by indicating a level of awakening of a human body, as well as expressing a concentration and an emotional level by illumination by sequentially computing and calculating the color temperature, illuminance, circadian action factor, and bio-illuminance.

FIG. 2 is a diagram for schematically describing a bio-illuminance measuring system according to an embodiment of the present disclosure.

Referring to FIG. 2, a bio-illuminance measuring system 100 may include an analog front-end chip 110, a transceiving device 120, and a bio-illuminance calculation server 130.

The analog front-end chip 110 may include a first photosensor 111 and a second photosensor 112. The first photosensor 111 and the second photosensor 112 may be embedded in the analog front-end chip 110. The analog front-end chip 110 may be an analog front-end chip.

The first photosensor 111 and the second photosensor 112 may be photodiodes. The first photosensor 111 and the second photosensor 112 may sense external light and generate a photocurrent corresponding thereto. The photocurrent may be proportional to the intensity of light. The first photosensor 111 and the second photosensor 112 may be linear photosensors.

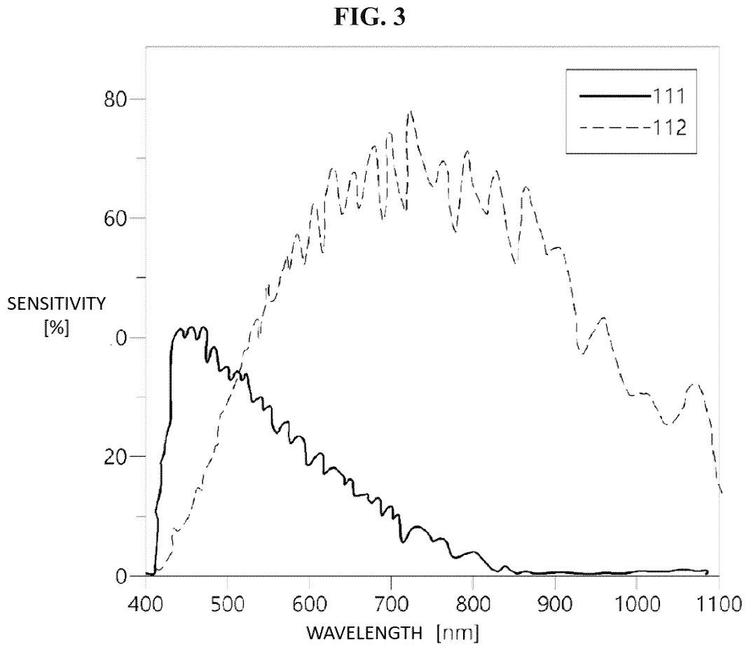

The first photosensor 111 and the second photosensor 112 may have different characteristics. The first photosensor 111 and the second photosensor 112 may have different sensitivities. The first photosensor 111 and the second photosensor 112 will be described in more detail below with reference to FIG. 3. The analog front-end chip 110 may convert optical voltages, corresponding to the first photosensor 111 and the second photosensor 112, respectively, into digital data DD1 and DD2, and output the digital data DD1 and DD2 to the transceiving device 120. The digital data DD1 and DD2 may have a digital value of at least 12 bits. For example, the digital data DD1 and DD2 may have a digital value of 14 bits.

The transceiving device 120 may receive the digital data DD1 and DD2 for the first photovoltage and the second photovoltage from the analog front-end chip 110. The transceiving device 120 may transmit the digital data DD1 and DD2 to the bio-illuminance calculation server 130 at a set speed. The transceiving device 120 may transmit the digital data DD1 and DD2 to a processor 131.

The transceiving device 120 may communicate wirelessly or wiredly. The transceiving device 120 may be configured to include at least one or more of USB, serial and parallel communication, RFID, infrared communication, Bluetooth, ZigBee, WiFi, UWB, near-field wired and wireless communication including WAV and NFC, wired and wireless communication means capable of voice and data communication, DAB, DAB+, AT-DMB, ATSC-M/W, digital radio broadcasting (including DRM, DRM+, and HD-Radio), broadcast receiving means including FM supplementary broadcasting, and combinations thereof.

The bio-illuminance calculation server 130 may include all devices capable of communicating with the transceiving device 120 wiredly or wirelessly. The bio-illuminance calculation server 130 may receive the digital data DD1 and DD2 for the first photovoltage and the second photovoltage from the transceiving device 120.

The bio-illuminance calculation server 130 in FIG. 2 may provide a computing resource for sequentially calculating a correlated color temperature (CCT), a circadian action factor (CAF), illuminance (Lux), and bio-illuminance (Biolux) using the digital data DD1 and DD2 for the first photovoltage and the second photovoltage as inputs.

The bio-illuminance calculation server 130 may be implemented as a cloud-based system, and may include various servers, apparatuses, devices, terminals, and the like therein. For example, the bio-illuminance calculation server 130 may include various types of servers, such as an application server, a control server, a data collection server, a data storage server, a data processing server, an application programming interface (API) providing server, a data display server, and a server for providing a specific function. The bio-illuminance calculation server 130 may be configured as a single system to perform processes such as data collection, data storage, data processing, API provision, data display, and the like. However, the bio-illuminance calculation server 130 is not limited thereto, and a plurality of servers may simultaneously process the processes.

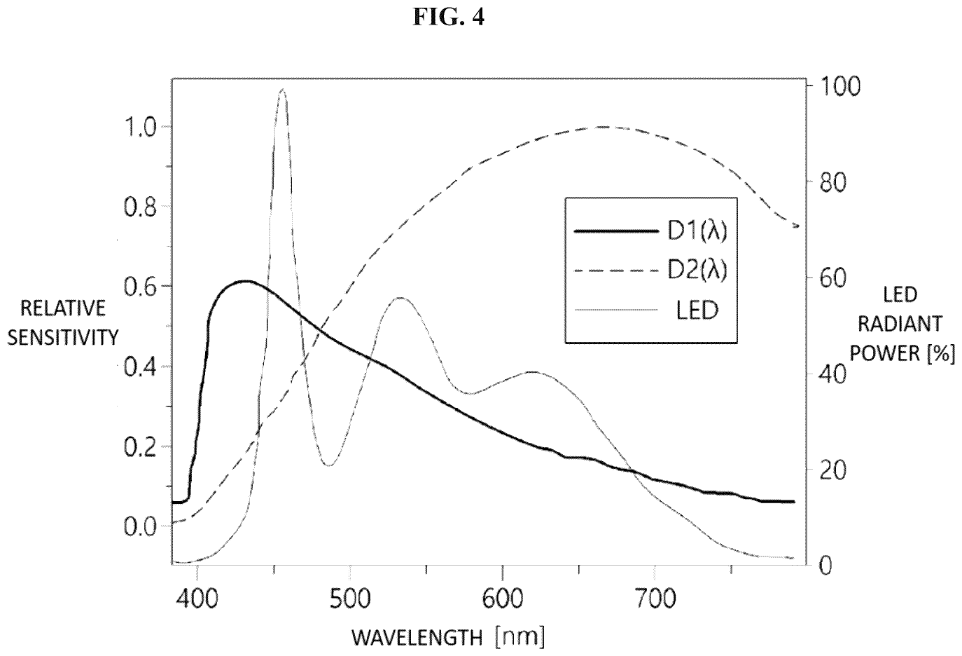

The bio-illuminance calculation server 130 may include at least one processor 131. The at least one processor 131 may sequentially calculate the correlated color temperature (CCT), the circadian action factor (CAF), the illuminance (Lux), and the bio-illuminance (Biolux) using the digital data DD1 and DD2 for the first photovoltage and the second photovoltage input through the transceiving device 120. The processor 131 may invoke a pre-stored function based on the first photovoltage and the second photovoltage. The at least one processor 131 may be configured to calculate the bio-illuminance and output the calculated bio-illuminance. An operation of the at least one processor 131 will be described in more detail below with reference to FIG. 4.

Although not illustrated in FIG. 2, the bio-illuminance measuring system 100 may further include an infrared filter configured to filter infrared components of external light.

FIG. 3 is a diagram illustrating light response characteristics of a photosensor according to an embodiment of the present disclosure.

Referring to FIG. 3, the first photosensor 111 of FIG. 2 and the second photosensor 112 of FIG. 2 may be photodiodes. FIG. 3 may illustrate characteristic curves of a first photosensor 111 in FIG. 2 and a second photosensor 112 in FIG. 2, in which light response characteristics are differently separated by using a shallow junction and a deep-well junction in a CMOS process.

FIG. 4 is a diagram illustrating a sensitivity curve of the photosensor according to an embodiment of the present disclosure.

Referring to FIG. 4, D1(λ) may represent a sensitivity curve of the first photosensor 111 in FIG. 2, and D2(λ) may represent a sensitivity curve of the second photosensor 112 in FIG. 2.

The sensitivity curve D1(λ) of the first photosensor may have a maximum sensitivity in a relatively short wavelength band. The sensitivity curve D1(λ) of the first photosensor may be similar to the circadian sensitivity curve A of FIG. 1. A peak wavelength of the first photosensor may be configured to be adjacent to a peak wavelength of the circadian sensitivity curve A in FIG. 1. The first photosensor may have a high sensitivity to wavelengths of a blue color system.

The sensitivity curve D2(λ) of the second photosensor may have a maximum sensitivity in a relatively long wavelength band. The sensitivity curve D2(λ) of the second photosensor may be similar to the visual sensitivity curve B in FIG. 1. The peak wavelength of the second photosensor may be configured to be adjacent to a peak wavelength of the visual sensitivity curve B in FIG. 1. The sensitivity curve D2(λ) of the second photosensor may be responsive to an infrared wavelength band. The second photosensor may have a high sensitivity to wavelengths of a red color system.

Since an LED lamp LED does not include an infrared (IR) component, the sensitivity curve D2(λ) of the second photosensor may be used to calculate an optical index for visible light without an error due to the infrared component. The LED lamp that does not include the infrared component may be appropriately used even though the second photosensor 112 in FIG. 2 has an infrared response characteristic, but in the case of an LED lamp that includes the infrared component, an infrared filter may be additionally required.

The photocurrents generated from the first photosensor 111 and the second photosensor 112, respectively may be converted into the photovoltages D1 and D2. The first photovoltage D1 may be calculated by Equation 1 below, and the second photovoltage D2 may be calculated by Equation 2 below.

$${D}_{1} = {K}_{1} {\int }_{380 nm}^{780 nm} {D}_{1} ( λ ) S ( λ ) d λ [ V ] \qquad [ Equation 1 ]$$

$${D}_{2} = {K}_{2} {\int }_{380 nm}^{780 nm} {D}_{2} ( λ ) S ( λ ) d λ [ V ] \qquad [ Equation 2 ]$$

D₁(λ) may mean the sensitivity of the first photosensor 111 in FIG. 2, D₂(λ) may means the sensitivity of the second photosensor 112 in FIG. 2, and S(λ) may mean a radiant flux of light. Sensitivity may be a performance index that indicates how efficiently the photosensor may convert light into electricity. The radiant flux of light may mean energy that is emitted or received in all directions through a certain area per unit time. In other words, the radiant flux of light may mean a temporal proportion of energy radiation. A unit of the radiant flux of light may be a watt.

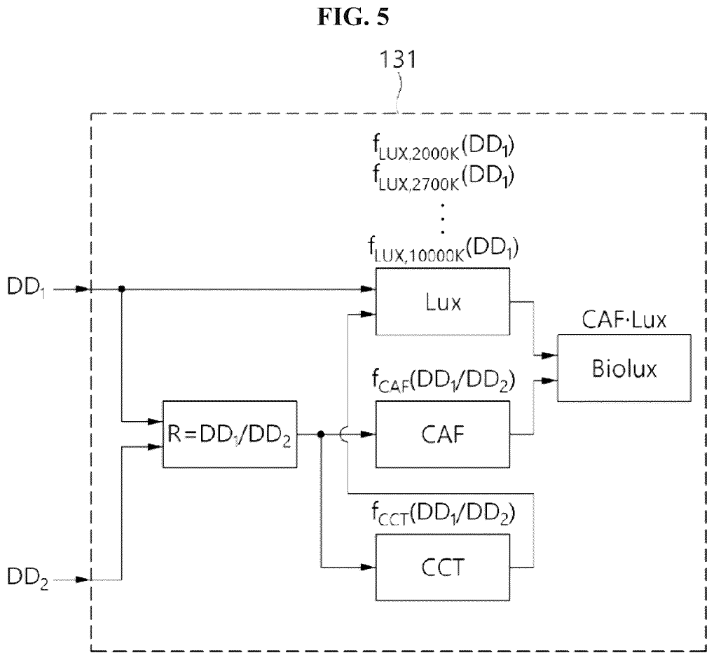

FIG. 5 is a diagram illustrating an operation of a bio-illuminance calculation server according to an embodiment of the present disclosure.

Referring to FIG. 5, the bio-illuminance calculation server 130 in FIG. 2 may receive the digital data DD1 and DD2 for the first photovoltage and the second photovoltage from the transceiving device 120 in FIG. 2. The at least one processor 131 of the bio-illuminance calculation server 130 in FIG. 2 may invoke a stored function. The at least one processor 131 may sequentially calculate the correlated color temperature (CCT), the circadian action factor (CAF), the illuminance (Lux), and the bio-illuminance (Biolux) using the digital data DD1 and DD2 for the first photovoltage and the second photovoltage as inputs.

The at least one processor 131 may calculate a voltage ratio R by dividing the digital data DD1 of the first photovoltage and the digital data DD2 of the second photovoltage. That is, the voltage ratio R may be a ratio of the digital data DD1 of the first photovoltage to the digital data DD2 of the second photovoltage. The at least one processor 131 may calculate the correlated color temperature (CCT) of the external light and the circadian action factor (CAF) of the external light by using the voltage ratio R, calculate the illuminance (Lux) based on the first photovoltage DD1 and the correlated color temperature (CCT), and calculate the bio-illuminance (Biolux) based on the illuminance (Lux) and the circadian action factor (CAF).

The circadian action factor (CAF) as a unique characteristic of the light source may be calculated according to Equation 3. The greater the circadian action factor (CAF), the greater the degree of awakening to the light source.

$$CAF = \frac{{K}_{c 0} {\int }_{380 nm}^{780 nm} C ( λ ) S ( λ ) d λ \div {\int }_{380 nm}^{780 nm} S ( λ ) d λ}{{K}_{0} {\int }_{380 nm}^{780 nm} V ( λ ) S ( λ ) d λ \div {\int }_{380 nm}^{780 nm} S ( λ ) d λ} \qquad [ Equation 3 ]$$

The at least one processor 131 may calculate the bio-illuminance (Biolux) using Equation 4.

$$Biolux = illuminance ( Lux ) \times circadian action factor ( CAF ) \qquad [ Equation 4 ]$$

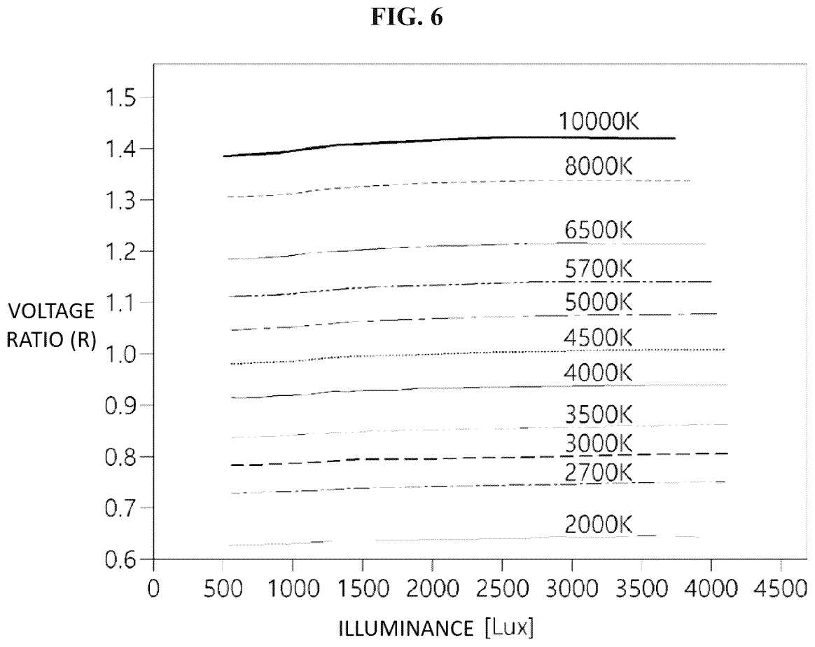

FIG. 6 is a diagram illustrating a color temperature and a voltage ratio in accordance with a change in illuminance according to an embodiment of the present disclosure.

Referring to FIG. 6, when the first photosensor 111 in FIG. 2 has high sensitivity to the wavelength of the blue color system and the second photosensor 112 in FIG. 2 has high sensitivity to the wavelength of the red color system, the voltage ratio R may exhibit a monotonically increasing characteristic with respect to the correlated color temperature (CCT). The voltage ratio R has low sensitivity to illuminance and may increase monotonically with respect to the color temperature.

The at least one processor 131 in FIG. 5 may obtain a polynomial type f_{CCT}(DD1/DD2) from a result of measuring a correlation between the voltage ratio R and the color temperature according to a change in illuminance. The at least one processor 131 may calculate the correlated color temperature (CCT) of the external light by using the voltage ratio R according to the change in illuminance.

A color temperature (CT) of illumination may affect human sensibility and awakening level. The color temperature expresses a color of the light source in numbers using an absolute temperature, and the color temperature is lower in the red color-system light source and higher in the blue color-system light source. Traditionally, Kelvin, which is an absolute temperature unit, is used, and since black body radiation is not performed in actual illumination, a correlated color temperature (CCT), which is an approximate value on a CIE chromaticity table, is used.

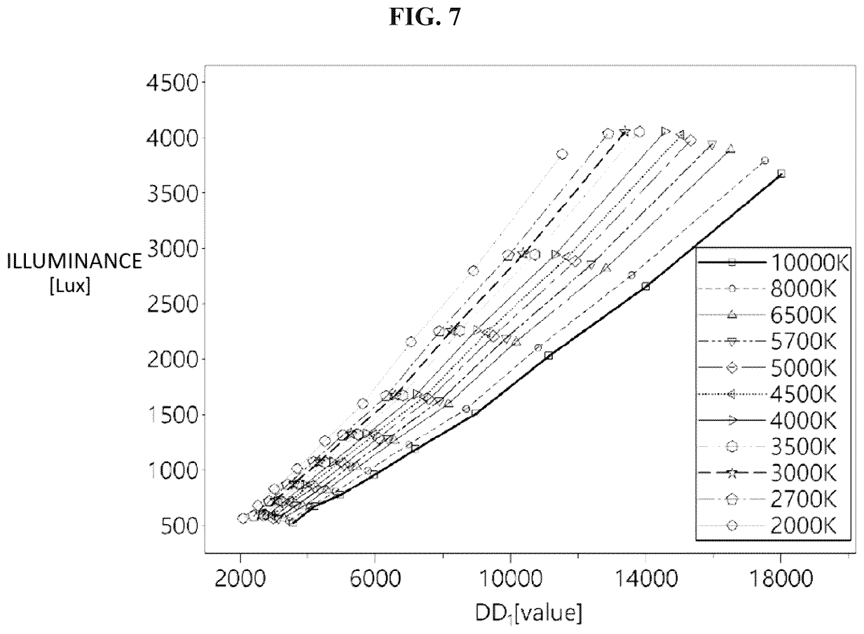

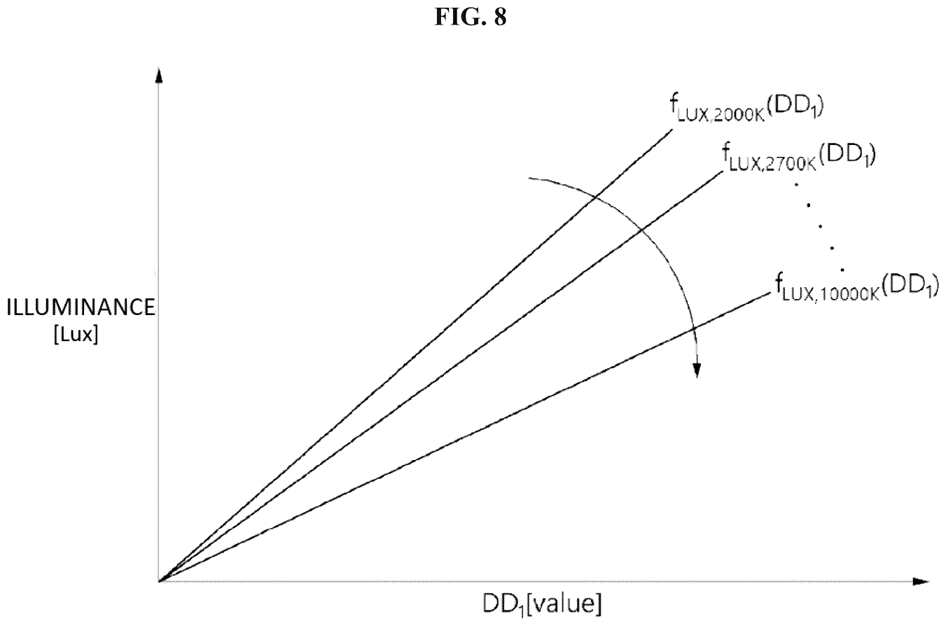

FIG. 7 is a diagram illustrating a correlation between a photovoltage and illuminance in accordance with the color temperature according to an embodiment of the present disclosure and FIG. 8 is a diagram illustrating a correlation function group of the photovoltage and the illuminance.

Referring to FIGS. 7 and 8, the digital data DD1 and the illuminance of the first photovoltage may have a monotonically increasing characteristic. The photovoltage of the first photosensor may be similar to a visual illuminance characteristic curve and may have no infrared (IR) reaction. Since the sensitivity curve D1(λ) in FIG. 4 of the first photosensor does not coincide with the visual illuminance characteristic curve, variation according to the color temperature may occur even for the same photovoltage value. From the measurement result of FIG. 7, a function group using the color temperature as a parameter may be constructed as illustrated in FIG. 8. From this, the illuminance may be calculated by selecting a corresponding function using the previously calculated color temperature as the parameter. In this case, Equation 5 below may be used.

$$LUX = {f}_{LUX , 2000 K} ( DD 1 ) , {f}_{LUX , 2700 K} ( DD 1 ) , , {f}_{LUX , 10000 K} ( DD 1 ) \qquad [ Equation 5 ]$$

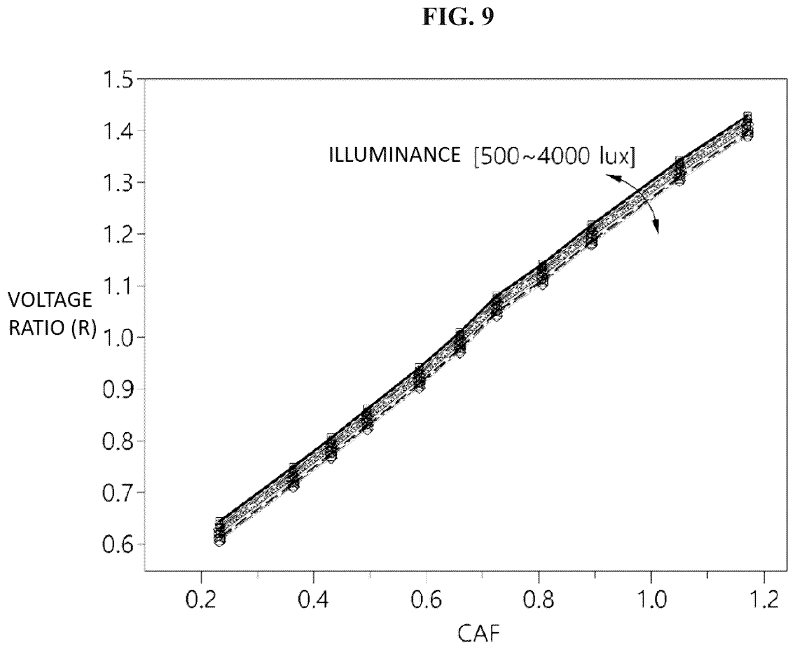

FIG. 9 is a diagram illustrating a correlation between a circadian action factor and a voltage ratio according to an embodiment of the present disclosure.

Referring to FIG. 9, the voltage ratio R may monotonically increase to be insensitive to the illuminance Lux with respect to the circadian action factor CAF. It can be seen that the circadian action factor CAF and the voltage ratio R have a linear correlation, from which f_{CAF}(DD1/DD2) is constructed. When the processor 141 in FIG. 2 invokes this function, the circadian action factor CAF in FIG. 5 may be calculated for the input of the voltage ratio R in FIG. 5.

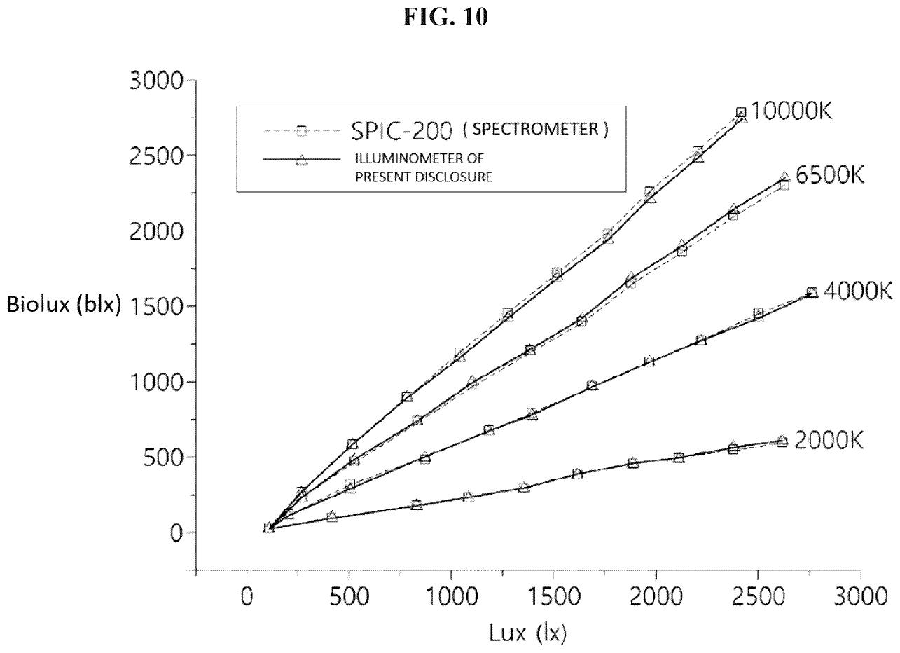

FIG. 10 is a diagram illustrating a correlation between bio-illuminance and illuminance in accordance with the color temperature according to an embodiment of the present disclosure.

Referring to FIG. 10, the precision of the bio-illuminance measuring apparatus and system of the present disclosure may be close to that of a spectrometer.

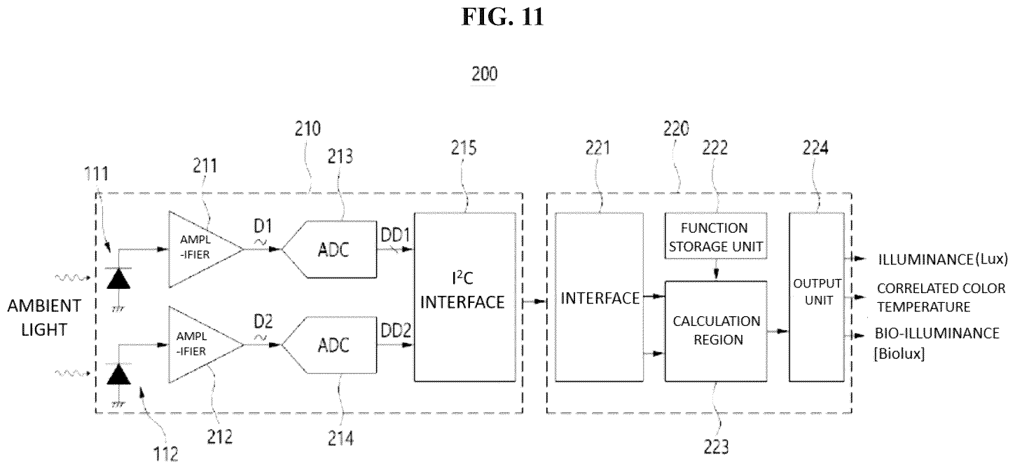

FIG. 11 is a diagram for schematically describing a bio-illuminance measuring system according to an embodiment of the present disclosure. FIG. 11 will be described with reference to FIG. 2, and the same reference numerals will be used with respect to the same components, and redundant descriptions will be omitted.

Referring to FIG. 11, a bio-illuminance measuring system 200 may include an analog front-end chip 210 and a bio-illuminance calculation server 220. The analog front-end chip 210 and/or the bio-illuminance calculation server 220 may be implemented in a personal computer (PC), a data server, or a hand-held device. The handheld device may be implemented as a laptop computer, a mobile phone, a smart phone, a tablet PC, a mobile internet device (MID), personal digital assistant (PDA), enterprise digital assistant (EDA), a digital still camera, a digital video camera, a portable multimedia player (PMP), a personal navigation device (PND), a hand-held game console, an e-book, or a smart device. The smart device may be implemented as a smart watch, a smart band, or a smart ring.

The analog front-end chip 210 may include a first optical sensor 111 and a second optical sensor 112. The analog front-end chip 210 may further include first and second amplifiers 211 and 212, first and second analog-to-digital converters 213 and 214, and an interface 215.

The first and second amplifiers 211 and 212 may amplify and convert the currents sensed by the first and second photosensors 111 and 112 into first and second photovoltages D1 and D2, respectively.

The first and second analog-to-digital converters 213 and 214 may receive the first and second photovoltages D1 and D2 and convert the received first and second photovoltages D1 and D2 into first and second digital data DD1 and DD2. The first and second digital data DD1 and DD2 may have a size of at least 12 bits. For example, the first and second digital data DD1 and DD2 may be 14 bits.

A path for outputting the first digital data DD1 from an output value of the first photosensor 111 and a path for outputting the second digital data DD2 from an output value of the second photosensor 112 may exist independently.

The interface 215 may be an I²C interface. The first and second digital data DD1 and DD2 may be transmitted to the bio-illuminance calculation server 220 through the interface 215.

The bio-illuminance calculation server 220 may include an interface 221, a function storage unit 222, a calculation region 223, and an output unit 224.

The interface 221 may be an I²C interface. The first and second digital data DD1 and DD2 may be received from the analog front-end chip 210 through the interface 221.

The function storage unit 222 may include functions for calculating the correlated color temperature (CCT) in FIG. 5, the circadian action factor (CAF) in FIG. 5, the illuminance (Lux) in FIG. 5, and the bio-illuminance (Biolux) in FIG. 5 described above with reference to FIGS. 5 to 10.

The calculation region 223 may calculate the voltage ratio R in FIG. 5 using the first and second digital data DD1 and DD2. The calculation region 223 may invoke any one of the functions stored in the function storage unit 222, and may calculate the correlated color temperature (CCT) in FIG. 5, the circadian action factor (CAF) in FIG. 5, the illuminance (Lux) in FIG. 5, and the bio-illuminance (Biolux) in FIG. 5 using the calculated voltage ratio R in FIG. 5.

The output unit 224 may output any one of the correlated color temperature (CCT) in FIG. 5, the circadian action factor (CAF) in FIG. 5, the illuminance (Lux) in FIG. 5, and the bio-illuminance (Biolux) in FIG. 5 calculated by the calculation region 223 to the outside of the bio-illuminance calculation server 220.

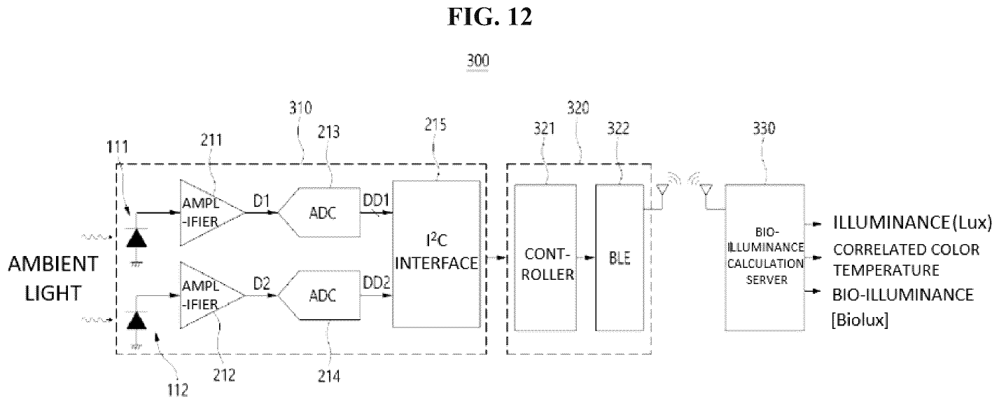

FIG. 12 is a diagram for schematically describing a bio-illuminance measuring system according to an embodiment of the present disclosure. FIG. 12 will be described with reference to FIGS. 2 and 11, and the same reference numerals will be used with respect to the same components, and redundant descriptions will be omitted.

Referring to FIG. 12, an analog front-end chip 310 of the bio-illuminance measuring system 300 may have the same configuration as the analog front-end chip 210 of FIG. 11. A bio-illuminance calculation server 330 of the bio-illuminance measuring system 300 may include the same components as those of the bio-illuminance calculation server 130 in FIG. 2 or the bio-illuminance calculation server 220 in FIG. 11. That is, the bio-illuminance measuring system 300 may further include a master chip 320, unlike FIGS. 2 and 11.

The master chip 320 may enable wireless communication between the analog front-end chip 310 and the bio-illuminance calculation server 330 of the bio-illuminance measuring system 300. The master chip 320 may include a controller 321 and a Bluetooth 322.

The controller 321 may control the first and second digital data DD1 and DD2 transmitted from the interface 215 of the analog front-end chip 310. The first and second digital data DD1 and DD2 may be I²C data.

The Bluetooth 322 may wirelessly output the first and second digital data DD1 and DD2. The Bluetooth 322 may wirelessly transmit the first and second digital data DD1 and DD2 to the bio-illuminance calculation server 330. The Bluetooth 322 may be low-power Bluetooth.

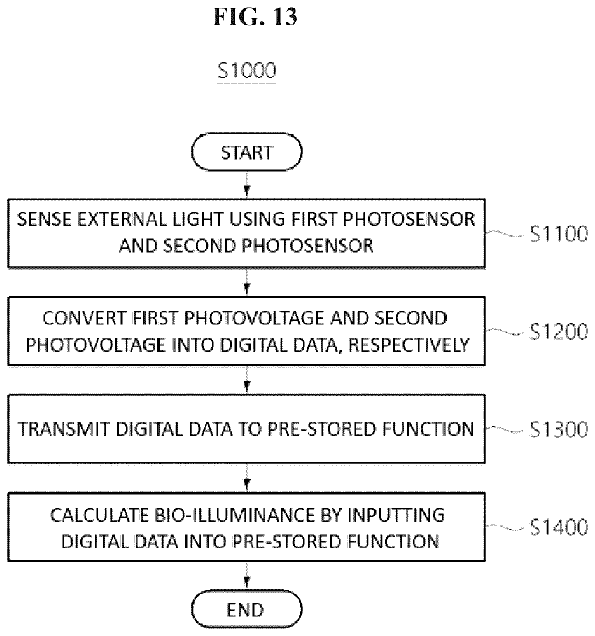

FIGS. 13 to 15 are flowcharts illustrating a bio-illuminance measurement method S1000 of the bio-illuminance measuring system according to an embodiment of the present disclosure. FIGS. 13 to 15 will be described with reference to FIGS. 1 to 12 described above, and redundant description of each configuration may be omitted.

Referring to FIG. 13, the bio illuminance measuring systems 100 in FIG. 2, 200 in FIG. 11, and 300 in FIG. 12 may sequentially perform steps S1100 to S1400.

In step S1100, the first photosensor 111 in FIG. 2 and the second photosensor 112 in FIG. 2 may sense external light. The first photosensor 111 in FIG. 2 and the second photosensor 112 in FIG. 2 may generate a photovoltage according to the intensity of light. The peak wavelengths of the first photosensor 111 in FIG. 2 and the second photosensor 112 in FIG. 2 may be configured to be adjacent to a peak wavelength of a curve of circadian sensitivity and a peak wavelength of the curve of visual sensitivity, respectively.

In step S1200, the first photovoltage and the second photovoltage generated by the first photosensor 111 in FIG. 2 and the second photosensor 112 in FIG. 2, respectively, may be converted into the digital data DD1 and DD2 in FIG. 2, respectively. According to an embodiment, an amplifier and an ADC may be used as in FIG. 11.

In step S1300, the bio-illuminance calculation server 130 in FIG. 2 may receive the digital data DD1 and DD2 in FIG. 2 for the first photovoltage and the second photovoltage, and may transmit the digital data DD1 and DD2 in FIG. 2 for the first photovoltage and the second photovoltage to the function pre-stored in the bio-illuminance calculation server 130 in FIG. 2. For example, the bio-illuminance calculation server 130 may transmit the digital data DD1 and DD2 in FIG. 11 for the first photovoltage and the second photovoltage to the function storage unit 222 in FIG. 11.

In step S1400, the bio-illuminance calculation server 130 in FIG. 2 may calculate the bio-illuminance by inputting the transmitted digital data DD1 and DD2 in FIG. 2 for the first photovoltage and the second photovoltage into the pre-stored function. For example, the calculation region 223 in FIG. 11 may calculate the bio-illuminance using the digital data DD1 and DD2 in FIG. 11 for the first photovoltage and the second photovoltage, and the pre-stored function invoked from the function storage unit 222.

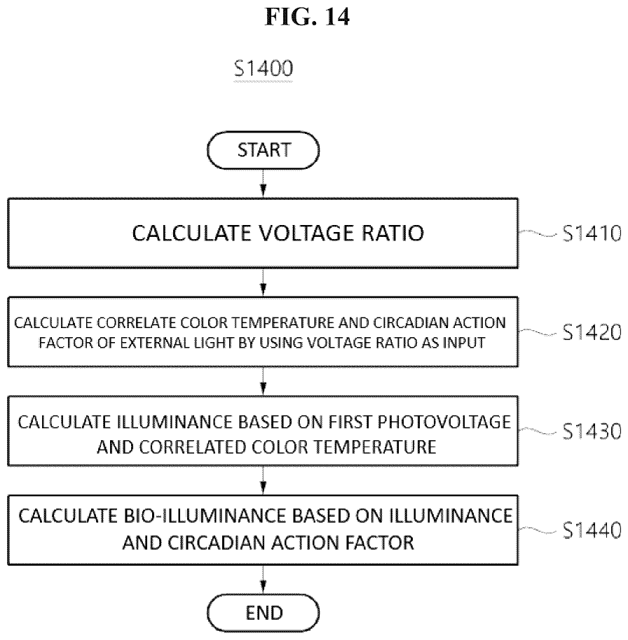

Referring to FIG. 14, the bio-illuminance measuring system 100 in FIG. 2, 200 in FIG. 11, and 300 in FIG. 12 may sequentially perform steps S1410 and S1440. Step S1400 in FIG. 13 may include steps S1410 and S1440.

In step S1410, the bio-illuminance calculation server 130 in FIG. 2 may calculate a voltage ratio R in FIG. 5 between the first photovoltage and the second photovoltage by using the digital data DD1 and DD2 in FIG. 2 for the first photovoltage and the second photovoltage. For example, the calculation region 223 in FIG. 11 may calculate the voltage ratio R in FIG. 5 using the digital data DD1 and DD2 in FIG. 11 for the first photovoltage and the second photovoltage.

In step S1420, the bio-illuminance calculation server 130 in FIG. 2 may calculate the correlated color temperature (CCT) in FIG. 5 of the external light and the circadian action factor (CAF) in FIG. 5 of the external light, using the voltage ratio R in FIG. 5 as an input. For example, the calculation region 223 in FIG. 11 may calculate the correlated color temperature (CCT) in FIG. 5 of the external light and the circadian action factor (CAF) in FIG. 5 of the external light, using the digital data DD1 and DD2 in FIG. 11 for the first photovoltage and the second photovoltage, and the pre-stored function invoked from the function storage unit 222. The voltage ratio R in FIG. 5 of the first photovoltage to the second photovoltage may increase monotonically with respect to the correlated color temperature (CCT) in FIG. 5 of the external light. The voltage ratio R in FIG. 5 of the first photovoltage to the second photovoltage may monotonically increase to be insensitive to the illuminance with respect to the circadian action factor (CAF) in FIG. 5, of the external light.

In step S1430, the bio illuminance calculation server 130 in FIG. 2 may calculate the illuminance based on the digital data DD1 in FIG. 5 for the first photovoltage and the calculated correlated color temperature (CCT) in FIG. 5. For example, the calculation region 223 in FIG. 11 may calculate the illuminance (Lux) in FIG. 5 using the digital data DD1 in FIG. 11 for the first photovoltage, the pre-calculated correlated color temperature (CCT) in FIG. 5 of the external light, and the pre-stored function invoked from the function storage unit 222.

In step S1440, the bio-illuminance calculation server 130 in FIG. 2 may calculate the bio-illuminance (Biolux) in FIG. 5 based on the calculated illuminance (Lux) in FIG. 5, and the calculated circadian action factor (CAF) in FIG. 5. For example, the calculation region 223 in FIG. 11 may calculate the bio-illuminance (Biolux) in FIG. 5 using the calculated illuminance (Lux) in FIG. 5, the calculated circadian action factor (CAF) in FIG. 5, and the pre-stored function invoked from the function store 222.

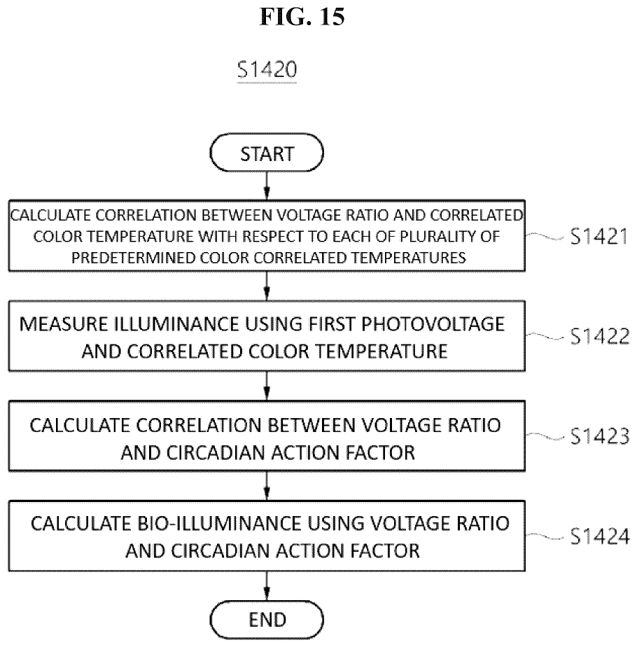

Referring to FIG. 15, the bio-illuminance measuring systems 100 in FIG. 2, 200 in FIG. 11, and 300 in FIG. 12 may sequentially perform steps S1421 to S1424. Step S1420 in FIG. 14 may include steps S1421 to S1424.

In step S1421, the bio-illuminance calculation server 130 in FIG. 2 may calculate a correlation between the voltage ratio R in FIG. 5 of the first photovoltage to the second photovoltage and the correlated color temperature for each of a plurality of predetermined correlated color temperatures. For example, the calculation region 223 in FIG. 11 may calculate the correlation between the voltage ratio R in FIG. 5 of the first photovoltage to the second photovoltage and the correlated color temperature for each of a plurality of predetermined correlated color temperatures. Each of the plurality of predetermined correlated color temperatures may be data stored in the function storage unit 222 in FIG. 11 and then invoked by the calculation region 223 in FIG. 11. The correlation of the voltage ratio R in FIG. 5 of the first photovoltage to the second photovoltage and the correlated color temperature may be stored in the function storage unit 222 in FIG. 11.

In step S1422, the bio-illuminance calculation server 130 in FIG. 2 may measure the illuminance using the first photovoltage and the correlated color temperature. The bio-illuminance calculation server 130 in FIG. 2 may measure the illuminance (Lux) in FIG. 5 using the digital data DD1 in FIG. 5 for the first photovoltage and the calculated correlated color temperature (CCT) in FIG. 5. For example, the calculation region 223 in FIG. 11 may measure the illuminance (Lux) in FIG. 5 using the digital data DD1 in FIG. 5 for the first photovoltage and the calculated correlated color temperature (CCT) in FIG. 5.

In step S1423, the bio-illuminance calculation server 130 in FIG. 2 may calculate a correlation between the voltage ratio R in FIG. 5 of the first photovoltage to the second photovoltage and the calculated circadian action factor (CAF) in FIG. 5. For example, the calculation region 223 in FIG. 11 may calculate the correlation between the voltage ratio R in FIG. 5 and the circadian action factor (CAF) in FIG. 5. The correlation between the voltage ratio R in FIG. 5 and the circadian action factor (CAF) in FIG. 5 may be stored in the function storage unit 222 in FIG. 11.

In step S1424, the bio-illuminance calculation server 130 in FIG. 2 may measure the bio-illuminance (Biolux) in FIG. 5 using the voltage ratio R in FIG. 5 of the first photovoltage to the second photovoltage and the calculated circadian action factor (CAF) in FIG. 5. For example, the calculation region 223 in FIG. 11 may calculate the bio-illuminance (Biolux) using the voltage ratio R in FIG. 5 and the circadian action factor (CAF) in FIG. 5.

Hereinabove, the embodiments of the present disclosure have been described with reference to the accompanying drawings, but those skilled in the art to which the present disclosure pertains can be understood that the present disclosure can be carried out in other detailed forms without changing the technical spirit or requisite features of the present disclosure. Therefore, it should be appreciated that the aforementioned embodiments are illustrative in all aspects and are not restrictive.

1. A system for measuring bio-illuminance, the system comprising:
an analog front-end chip including a first photosensor and a second photosensor;
a transceiving device; and
at least one processor configured to calculate bio-illuminance,
wherein the analog front-end chip is configured to sense external light and output a first photovoltage and a second photovoltage as digital data, respectively, using the first photosensor and the second photosensor,
wherein the transceiving device is configured to wirelessly or wiredly transmit the digital data of the first photovoltage and the second photovoltage to the processor, and
wherein the at least one processor is configured to calculate the bio-illuminance and output the calculated bio-illuminance by invoking a pre-stored function based on the first photovoltage and the second photovoltage.

2. The system for measuring bio-illuminance of claim 1, wherein peak wavelengths of the first photosensor and the second photosensor are configured to be adjacent to a peak wavelength of a curve of circadian sensitivity and a peak wavelength of a curve of visual sensitivity, respectively.

3. The system for measuring bio-illuminance of claim 1, wherein the at least one processor is configured to calculate a ratio of the first photovoltage and the second photovoltage, calculate a correlated color temperature of the external light and a circadian action factor of the external light using the ratio, calculate illuminance based on the first photovoltage and the correlated color temperature, and calculate bio illuminance based on the illuminance and the circadian action factor.

4. The system for measuring bio-illuminance of claim 1, wherein the transceiving device is configured to receive the digital data for the first photovoltage and the second photovoltage from the analog front-end chip and output the digital data wirelessly or wiredly at a set speed to a system in which the processor is embedded.

5. The system for measuring bio-illuminance of claim 3, wherein the ratio of the first photovoltage and the second photovoltage monotonically increases with respect to the correlated color temperature.

6. The system for measuring bio-illuminance of claim 3, wherein the ratio of the first photovoltage and the second photovoltage monotonically increases to be insensitive to the illuminance with respect to the circadian action factor.

7. The system for measuring bio-illuminance of claim 1, further comprising:
an infrared filter configured to filter an infrared component of the external light.

8. A method for measuring bio-illuminance, the method comprising:
sensing external light using a first photosensor and a second photosensor;
converting a first photovoltage and a second photovoltage respectively generated by the first photosensor and the second photosensor into digital data, respectively;
transmitting the digital data for the first photovoltage and the second photovoltage to a pre-stored function; and
calculating bio-illuminance by inputting the transmitted first photovoltage and second photovoltage data into the pre-stored function.

9. The method for measuring bio-illuminance of claim 8, wherein peak wavelengths of the first photosensor and the second photosensor are configured to be adjacent to a peak wavelength of a curve of circadian sensitivity and a peak wavelength of a curve of visual sensitivity, respectively.

10. The method for measuring bio-illuminance of claim 8, further comprising:
calculating a ratio of the first photovoltage and the second photovoltage;
calculating a correlated color temperature of the external light and a circadian action factor of the external light using the ratio as an input;
calculating an illuminance based on the first photovoltage and the correlated color temperature; and
calculating bio-illuminance based on the illuminance and the circadian action factor.

11. The method for measuring bio-illuminance of claim 10, wherein the ratio of the first photovoltage and the second photovoltage monotonically increases with respect to the correlated color temperature.

12. The method for measuring bio-illuminance of claim 10, wherein the ratio of the first photovoltage and the second photovoltage monotonically increases to be insensitive to the illuminance with respect to the circadian action factor.

13. The method for measuring bio-illuminance of claim 10, further comprising:
calculating a correlation between the ratio of the first photovoltage and the second photovoltage and the correlated color temperature for each of a plurality of predetermined correlated color temperatures;
measuring the illuminance using the first photovoltage and the correlated color temperature;
calculating a correlation between the ratio of the first photovoltage and the second photovoltage and the circadian action factor; and
measuring the bio-luminance using the ratio of the first photovoltage and the second photovoltage and the circadian action factor.

14. An apparatus for measuring bio-illuminance, the apparatus comprising:
an analog front-end chip including a first photosensor and a second photosensor having different light response characteristics, and configured to output digital data for a first photovoltage and a second photovoltage using the first photosensor and the second photosensor;
at least one processor configured to calculate bio-illuminance; and
a transceiving device configured to wirelessly or wiredly transmit the digital data for the first photovoltage and the second photovoltage to the processor,
wherein the at least one processor is configured to calculate a ratio of the first photovoltage and the second photovoltage, calculate a correlated color temperature of the external light and a circadian action factor of the external light based on the ratio, calculate illuminance based on the first photovoltage and the correlated color temperature, and calculate bio illuminance based on the illuminance and the circadian action factor.
